(19) United States
(12) Patent Application Publication
(10) Pub. No.: US 2004/0009181 A1
(43) Pub. Date: Jan. 15, 2004

(54) Treatment of ophthalmic conditions

(76) Inventor: James M. Lipton, Woodland Hills, CA (US)

Correspondence Address: PERKINS COIE LLP, POST OFFICE BOX 1208, SEATTLE, WA, 98111-1208, US

(21) Appl. No.: 10/298,142

(22) Filed: Nov. 15, 2002

Related U.S. Application Data
(60) Provisional application No. 60/382,887, filed on May 21, 2002.

(51) Int. Cl. A61K 39/00; A61K 39/38

(52) U.S. Cl. 424/184.1

(57) ABSTRACT

The present invention discloses a method of treating ophthalmic conditions by administering to a vertebrate inflicted with the condition a therapeutically effective amount of a peptide which is derived from alpha-melanocyte-stimulating hormone (α-MSH) and biologically functional equivalents thereof. Specifically, the peptides derived from alpha-melanocyte-stimulating hormone (α-MSH) include α-MSH (1-13) which is SYSMEHFRWGKPV (SEQ. ID NO. 4), α-MSH (4-10) which is MEHFRWG (SEQ. ID NO. 2), α-MSH (6-13) which is HFRWGKPV (SEQ. ID NO. 3), α-MSH (11-13) which is KPV (SEQ. ID NO. 1), and a KPV dimer (SEQ. ID NO. 5). The ophthalmic condition can be the result of an on going insult such as “Computer Eyes” or an acute or chronic infection of the eyes. The infective organism can be caused by a microorganism, which includes a bacteria, a fungi or a virus. The vertebrate includes a bird and a mammal. The peptide has antipyretic, anti-inflammatory, anti-bacterial, antifungal, and antiviral properties and therefore can be administered at the onset of the ophthalmic condition before the insult causing the condition is determined as well as thereafter.

### CLAIM OF PRIORITY

The present application is a continuation-in-part of U.S. patent application Ser. No. 10/382,887.

### FIELD OF INVENTION

The present invention relates to the treatment of ophthalmic conditions using antimicrobial, anti-inflammatory peptides; specifically, α-MSH peptide formulations.

### BACKGROUND OF THE INVENTION

Eyes are the windows of a body, open to the external world and rich in nutrients, helping a living being to perceive the surrounding environment. In constant use in waking hours, eyestrain and fatigue are common. Recent working environments create increased fatigue and strain on the eyes due to the amount of time working with computer monitors, florescent lighting and disparate lighting in offices compared to incoming sunlight through windows. The consequence of long term eye fatigue and strain is inflammation in the eyes and blurred vision.

Additionally, the eyes are vulnerable to sources of inflammation such as pollens, dust, elemental conditions and virulent microorganisms, the invasion and uncontrolled growth of which cause various types of ophthalmic infections, for instance, blepharitis, conjunctivitis and keratitis.

A relatively recent term for Computer Vision Syndrome (CVS) has gained acceptance. “Computer Eyes,” now describes what is and had been an increasingly common diagnosis for eyestrain associated with use of computer monitors. CVS has been diagnosed in millions of cases in the United States. (Bremer Communications, 2000). A recent study by the National Institute of Heath and Safety disclosed that as many as 90% of employees who work with computers for more than three hours a day suffer from a variety of eye pathologies. Id. Often the conditions are directly related to muscle strain. The sufferer will usually not complain of “muscle strain of the eyes” but of the inflammatory sequlae noted by reduced blinking and dryness, erythema and blurred vision.

Treatments for CVS have ranged from nutritional supplements, ergonomic exercises, eyeglasses and, of course, eye drops. Natural treatments for the eyes are best as they avoid complications associated with side effects. However, even natural treatments, like euphrasia (the scientific name of the plant, eyebright) for example, may be intolerable to many due to allergies. Further, the treatments associated with eye drops must be repeated as often as every two hours. Any increase in duration of action of CVS treatments will be beneficial. Further, an eye drop that combined antimicrobial efficacy with anti-inflammatory efficacy with limited side effects is needed.

Antimicrobial/anti-inflammatory eye drops are currently in use for a range of eye infections. The common types of microorganisms causing ophthalmic infections are viruses, bacteria, and fungi. These microorganisms may directly invade the surface of the eye, or permeate into the globe of the eye through trauma or surgery, or transmit into the eye through the blood stream or lymphatic system as a consequence of a systemic disease. The microorganisms may attack any part of the eye structure, including the conjunctiva, the cornea, the uvea, the vitreous body, the retina, and the optic nerve.

Ophthalmic infections can cause severe pain, swollen and red tissues in or around eyes, and blurred or decreased vision and warrant immediate medical treatment. Before an appropriate culture and sensitivity of the microorganism causing the infections is performed, initial treatment options are usually very limited. Further complicating this picture is the nature of the infection itself. Differentiation of a bacteria-caused ophthalmic infection from virus-caused or fungi-caused infection on the basis of clinical observation is frequently not reliable. Leibowitz et al., Human Conjunctivitis: Diagnostic Evaluation, Arch. Ophthalmol. 94: 1747-1749 (1976). Once the culture and sensitivity data are available, usually days after culture of the infections, the condition can then be treated with pertinent antimicrobial agents, including antiviral agents, antibacterial agents or antifungal agents, individually or in combination.

The antiviral agents commonly used in treating ophthalmic infections are Idoxurine and Acyclovir. Idoxurine inhibits the replication of viral DNA and is effective against Herpes simplex virus (HSV). However, studies have shown that Idoxurine is toxic to corneal epithelial cells and treatment over a little as seven days may cause punctate lesions to develop. Lazarus et al., An in vitro Method Which Assesses Corneal Epithelial Toxicity due to Antineoplastic, Preservative and Antimicrobial Agents, Lens Eye Toxic. Res. 6: 59-85 (1989). Acyclovir inhibits DNA replication of Herpes zoster virus (HZV) and therefore is commonly used in HZV-caused blepharitis or keratitis. However, it has been reported that Acyclovir has little or no preventative effect on ocular complications of HZV. Aylad et al., Influence of Oral Acyclovir on Ocular Complications of Herpes zoster ophthalmicus, Eye 8: 70-74 (1994).

The majority of bacteria-caused ophthalmic infections are treated with topically applied ophthalmic antibacterial agents, including sulfonamide, tetracycline, chloramphenicol, aminoglycoside, beta-lactam, vancomycin, and fluoroquinolone. Leeming, Treatment of Ocular Infections with Topical Antibacterials, Clin. Pharmacokinet. 37: 351-360 (1999). However, considerable resistance to the antibacterial agents has been reported. Studies have shown that 75% of ocular Staphylococcus species are resistant to tetracycline. Doughtery et al., The Role of Tetracycline in Chronic Blepharitis: Inhibition of Lipase Production in Staphylococcus, Inv. Ophthalmol. Vic. Sci. 32: 2970-2975 (1991). Flavobacterium indologenes is now resistant to most antibacterial agents. Lu & Chan, Flavobacterium indologenes Keratitis, Ophthalmologica 211: 98-100 (1997). Streptococcus pneumoniae and some strains of pneunococcus are resistant to penicillin. Wilkins et al., Penicillin-Resistant Streptococcus pneumoniae Keratitis, Cornea 15: 99-100 (1996). Approximately one third of Staphylococcus strains are resistant to gentamycin. Huberspitz et al., Corneal Ulceration: An Update from a Specialized Ambulatory Care Centre, Klinische. Monals. Augen. 200: 251-256 (1992). Additionally, 50% of bacteria isolated from corneal ulcers are resistant to all the common antibacterial agents. Satpathy & Vishalakshi, Ulcerative Keratitis: Microbial Profile and Sensitivity Pattern: A Five Year Study, Ann. Ophthalmol. Glaucoma 27: 301-306 (1995).

Antifungal agents are classified into two groups: polyenes such as amphotericin-B and azoles such as fluconazole. Amphotericin B remained the drug of choice for many fungal infections. Amphotericin-B, however, was reported to have poor penetration into ocular tissues and show toxicity against eyes. Ishibashi & Kaufman, The Effects of Subconjunctival Miconozole in the Treatment of Experimental Candida Keratitis in Rabbits, Arch. Ophthalmol. 103: 1570-1573 (1985). Further, resistance to azoles and amphotericin-B has also been reported. Armstrong, The microbiology of the Eye, Ophthal. Physiol. Opt. 20: 429-503 (2000).

Given that toxicity and resistance are commonly associated with the treatment of ophthalmic infections using the existing antimicrobial agents, it is desirable to provide a method for the treatment of ophthalmic infections using an inventive antimicrobial agent to reduce or minimize the toxicity and resistance. It is also desirable to provide a method for the treatment of an ophthalmic infection using an inventive antimicrobial agent that has antiviral, antibacterial and antifungal properties and can be used to treat the ophthalmic infection immediately after the onset of the infection without first taking days to determine the nature of microorganism causing the infection. These treatments, as well as new treatments for inflammation of the eyes, are needed.

### SUMMARY OF INVENTION

The primary aspect of the present invention is directed to a method of treating ophthalmic conditions in a vertebrate inflicted with the condition which comprises administering pharmacologically effective amount of a peptide to the vertebrate. Specifically, infections, xerosis blepharitis, keratitis, CVS, eye strain and/or fatigue and inflammations of the eyes.

According to one embodiment of the invention, a peptide is selected from the group of peptides with an amino acid sequence consisting of KPV (SEQ. ID NO. 1), MEHFRWG (SEQ. ID NO. 2), HFRWGKPV (SEQ. ID NO. 3), SYSMEHFRWGKPV (SEQ. ID NO. 4), VPKC-s-s-CKPV (the “KPV dimer”) (SEQ. ID NO 5) and biologically functional equivalents thereof. The peptide may be administered through a conjunctival administration, a nasal administration, a buccal administration, an oral administration, a rectal administration, a vaginal administration, an epidermal administration, and a parenteral administration.

According to another embodiment of the invention, the peptide can be administered at the onset of the ophthalmic infection before a microorganism causing the ophthalmic infection is determined or after the microorganism causing the ophthalmic infection is determined. Similarly, the peptide may be administered prophylactically in to prevent xerosis, CVS and other inflammations of the eyes.

According to another embodiment of the invention, the peptide can be administered individually or with another peptide, or with an existing medicinal agent, or with a non-medicinal agent.

### BRIEF DESCRIPTION OF THE DRAWINGS

The accompanying figures and drawings are incorporated into and form a part of the specification to provide illustrative examples of the present invention and to explain the principles of the invention. The references cited are incorporated by reference as if fully set forth herein. The figures and drawings are only for purposes of illustrating preferred and alternate embodiments of how the invention can be made and used. It is to be understood, of course, that the drawing is intended to represent and illustrate the concepts of the invention. The figures and drawings are not to be construed as limiting the invention to only the illustrated and described examples. Various advantages and features of the present invention will be apparent from a consideration of the written specification and the accompanying figures and drawing.

FIG. 1 illustrates the anatomy of a human eye. The human eye structure includes conjunctiva, cornea, vitreous body, retina, and optic nerve.

FIG. 2 illustrates the effect of α-MSH [1-13] (SYSMEHFRWGKPV) or α-MSH [11-13] (KPV) on p24 release by TNF-α stimulated U1 cells. Both α-MSH [1-13] and KPV peptides inhibited p24 release over a broad spectrum of concentrations. In this and following figures, columns or dots represent the mean and bars represent the standard deviation or confidence interval when p<0.05 (*) or p<0.01 (**).

FIG. 3 illustrates the effect of KPV on RT and p24 release by stimulated U1 cells. Treatment with KPV (10⁻⁵M) inhibited HIV reverse transcriptase (RT) and p24 release from U1 cells exposed to different stimuli.

FIG. 4 illustrates the effect of KPV on HIV RNA in resting and PMA-stimulated U1 cells. Addition of KPV (10⁻⁵M) reduced by approximately 50% both spliced and unspliced HIV-1 RNA in PMA-stimulated U1 cells.

FIG. 5 illustrates the effect of α-MSH [1-13] α-MSH [11-13] and the “KPV dimer” (VPKC-s-s-CKPV) on S. aureus colony forming units (“CFU”) compared to controls. All three molecules significantly decreased S. aureus colony forming units over a broad range of peptide concentrations.

FIG. 6 illustrates the effect of α-MSH [1-13] α-MSH [11-13], and the “KPV dimer” on S. aureus colony forming units when S. aureus' growth is enhanced by urokinase. The treatment with urokinase increases S. aureus colony formation, but that the addition of α-MSH [1-13] or α-MSH [11-13] (KPV) significantly inhibited this urokinase-enhancing effect.

FIG. 7 illustrates the effect of α-MSH [1-13], α-MSH [11-13], and the “KPV dimer” which is VPKC-s-s-CKPV on C. albicans colony forming units (“CFU”) compared to controls. All three molecules significantly decreased C. albicans colony forming units over a broad range of peptide concentrations.

FIG. 8 represents a comparison of antifungal activity of certain peptides and fluconazole (all 10⁻⁶M). The most effective of the peptides were those including the C-terminal amino acid sequence of α-MSH, for example, α-MSH [1-13], α-MSH [6-13], and α-MSH [11-13].

FIG. 9 illustrates a molecular conformational structure of the KPV dimer, VPKC-s-s-CKPV. Molecular modeling study revealed the conformational structure of the KPV dimer. VPKC-s-s-CKPV adopts a like-β-tum-structure well organized and stabilized by intra-molecular hydrogen bounds. The tertiary structure of the dimer is folded and amino acids are well protected. It also resembles a cyclic peptide with a beta-turn.

### DETAILED DESCRIPTION OF THE INVENTION

The broadest aspect of the invention is a method for treating an ophthalmic infection using a peptide. A preferred embodiment of the invention is a method for treating an ophthalmic infection using a peptide selected from the group of peptides with an amino acid sequence consisting of KPV (SEQ. ID NO. 1, MEHFRWG (SEQ. ID NO. 2), HFRWGKPV (SEQ. ID NO. 3), SYSMEHFRWGKPV (SEQ. ID NO. 4), a KPV dimer VPKC-s-s-CKPV (SEQ. ID NO. 5), and biologically functional equivalents thereof.

SYSMEHFRWGKPV (SEQ. ID NO. 4) is the entire amino acid sequence of alpha-Melanocyte Stimulating Hormone (here on referred to as “α-MSH” or “alpha-MSH” or “α-MSH [1-13]”) and the first 13 amino acid sequence derived from Adrenocorticotropic Hormone (here on referred to as “ACTH”). U.S. Pat. No. 5,028,592 discloses that α-MSH (SEQ. ID NO. 4) has anti-pyretic and anti-inflammatory properties and that α-MSH[11-13] (SEQ. ID NO. 1), the C-terminal tripeptide is responsible for the anti-pyretic and anti-inflammatory properties of α-MSH. Since α-MSH and KPV are well disclosed and characterized in U.S. Pat. No. 5,028,592, this reference is hereby incorporated by reference in its entirety.

HFRWGKPV (SEQ. ID NO. 2) is the amino acid sequence of α-MSH from residue 6 through residue 13, which is also referred to as α-MSH[6-13]. MEHFRWG (SEQ. ID NO. 2) is the amino acid sequence of α-MSH from residue 4 through residue 10, which is also referred to as (α-MSH[4-10]. U.S. patent application Ser. No. 09/533,341, entitled “Antimicrobial and anti-inflammatory peptides for use in human immunodeficiency virus,” disclosed that peptides KPV (SEQ ID NO. 1), MEHFRWG (SEQ ID NO. 2), HFRWGKPV (SEQ ID NO. 3), and SYSMEHFRWGKPV (SEQ ID NO. 4) have anti-viral, anti-bacterial and anti-fungal properties. U.S. patent application Ser. No. 09/533,341 is hereby incorporated by reference in its entirety.

The KPV dimer is formed when the N-terminals of two KPV peptides are linked by a linker. For example, VPKC-s-s-CKPV (SEQ. ID NO. 5), one kind of the KPV dimer, is formed by adding a cysteine at the N-terminal of KPV peptide and allowing the cysteines of two CKPV peptides to form a disulfide bond (-s-s-). In other words, VPKC-s-s-CKPV is formed when a -Cys-s-s-Cys-linker links two KPV peptides. The linker can be modified to any kind of chemical bond that links the N-terminals of two KPV peptides together. The different variations of linkers create a modified KPV dimer. Preferred modified KPV dimer linkages may be selected from the group consisting of -Cys-s-s-Cys-, -DCys-s-s-Cys-, -Pen-s-s-Cys-, -Pen-s-s-DCys-, -DPen-s-s-Cys-, -DPen-s-s-DCys-, -DPen-s-s-DPen-, -Pen-s-s-Pen-, -hCys-s-s-Cys-, -hCys-s-s-DCys-, -hCys-s-s-hCys-, -DhCys-s-s-DhCys-, -DhCys-s-s-hCys-, -hCys-s-s-Pen-, -hCys-s-s-DPen-, or -DhCys-s-s-DPen-. It is more preferred that the linker be -Cys-Cys-. The term “Pen” refers to Penicillamine. The Term “Cys” refers to Cysteine. The Term “hCys” refers to Omocysteine. The prefix “D” refers to the dextro-form of an amino acid. Accordingly, it is preferred that the KPV dimer be VPK-Cys-s-s-Cys-KPV (SEQ ID NO. 5), VPK-DCys-s-s-Cys-KPV (SEQ ID NO. 6), VPK-Pen-s-s-Cys-KPV (SEQ ID NO. 7), VPK-Pen-s-s-DCys-KPV (SEQ ID NO. 8), VPK-DPen-s-s-Cys-KPV (SEQ ID NO. 9), VPK-DPen-s-s-DCys-KPV (SEQ ID NO. 10), VPK-DPen-s-s-DPen-KPV (SEQ ID NO. 11), VPK-Pen-s-s-Pen-KPV (SEQ ID NO. 12), VPK-hCys-s-s-Cys-KPV (SEQ ID NO. 13), VPK-hCys-s-s-DCys-KPV (SEQ ID NO. 14), VPK-hCys-s-s-hCys-KPV (SEQ ID NO. 15), VPK-DhCys-s-s-DhCys-KPV (SEQ ID NO. 16), VPK-DhCys-s-s-hCys-KPV (SEQ ID NO. 17), VPK-hCys-s-s-Pen-KPV (SEQ ID NO. 18), VPK-hCys-s-s-DPen-KPV (SEQ ID NO. 19), or VPK-DhCys-s-s-DPen-KPV (SEQ ID NO. 20). It is more preferred that the KPV dimer be VPK-Cys-s-s-Cys-KPV (SEQ ID NO. 5).

The biological functional equivalent is defined as an amino acid sequence that is functionally equivalent to KPV (SEQ. ID NO. 1), VPKC-s-s-CKPV (SEQ. ID NO. 5), MEHFRWG (SEQ. ID NO. 2), HFRWGKPV (SEQ. ID NO. 3), SYSMEHFRWGKPV (SEQ. ID NO. 4) and the KPV dimer in terms of biological activity. Although the specific amino acid sequences described here are effective, it is clear to those familiar with the art that amino acids can be substituted in the amino acid sequence or deleted without altering the effectiveness of the peptides. For example, modifications of the KPV dimer, some of which are described above, are biologically equivalent. Further, it is known that stabilization of the α-MSH sequence can greatly increase the activity of the peptide and that substitution of D-amino acid forms for L-forms can improve or decrease the effectiveness of peptides. For example, a stable analog of α-MSH, [Nle⁴ DPhe⁷]-α-MSH, which is known to have marked biological activity on melanocytes and melanoma cells, is approximately ten times more potent than the parent peptide in reducing fever. Holdeman, M. and Lipton, J. M., Antipyretic Activity of a Potent α-MSH Analog, Peptides 6, 273-5 (1985). Further, adding amino acids to the C-terminal α-MSH (11-13) sequence can reduce or enhance antipyretic potency (Deeter, L. B.; Martin, L. W.; Lipton, J. M., Antipyretic Properties of Centrally Administered α-MSH Fragments in the Rabbit, Peptides 9, 1285-8 (1989). Addition of glycine to form the 10-13 sequence slightly decreased potency; the 9-13 sequence was almost devoid of activity, whereas the potency of the 8-13 sequence was greater than that of the 11-13 sequence. It is known that [D-K¹¹] α-MSH 11-13 has the same general potency as the L-form of the tripeptide (α-MSH 11-13. Hiltz, M. E.; Catania, A.; Lipton, J. M., Anti-inflammatory Activity of α-MSH (11-13) Analogs: Influences of Alterations in Stereochemistry, Peptides12, 767-71 (1991). However, in one study, substitution with D-proline in position 12 of the tripeptide rendered it inactive. Substitution with the D-form of valine in position 13 or with the D-form of lysine at position 11 plus the D-form of valine at position 13 resulted in greater anti-inflammatory activity than with the L-form tripeptide. These examples indicate that alterations in the amino acid characteristics of the peptides can influence activity of the peptides or have little effect, depending upon the nature of the manipulation.

It is also understood that biological functional equivalents may be obtained by substitution of amino acids having similar hydropathic values. Thus, for example, isoleucine and leucine, which have a hydropathic index +4.5 and +3.8, respectively, can be substituted for valine, which has a hydropathic index of +4.2, and still obtain a protein having like biological activity. Alternatively, at the other end of the scale, lysine (−3.9) can be substituted for arginine (−4.5), and so on. In general, it is believed that amino acids can be successfully substituted where such amino acid has a hydropathic score of within about +/−1 hydropathic index unit of the replaced amino acid. The antimicrobial properties of biological functional equivalents can be measured through their inhibitory effect on the colony forming units in bacteria or fungi, or through their inhibitory effect on virus expression or transcription, as disclosed in the examples of this invention.

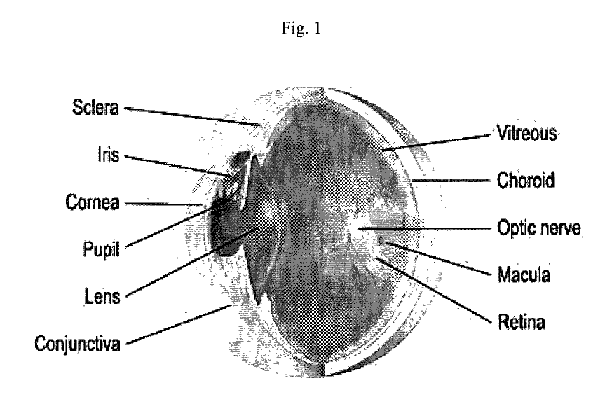

The terms “ophthalmic infection” used for this invention refer to an infection caused by a microorganism or microorganisms in or around an eye or the eye structure which include the eyelids and lacrimal apparatus, the conjunctiva, the cornea, the uvea, the vitreous body, the retina, and the optic nerve. See, FIG. 1. Ophthalmic infections include bacterial ophthalmic infections, fungal ophthalmic infections and viral ophthalmic infections.

Ophthalmic conditions contemplated in this invention include but are not limited to CVS (“Computer Eyes”) blepharitis, hordeolum, preseptal cellulitis, dacryocystitis, orbital cellulitis, erysipelas, vernal keratoconjunctivitis, bacterial conjunctivitis, conjunctival laceration, superior limbic keratoconjunctivitis, conjunctivitis with pseudomembrane, epidemic keratoconjunctivitis, bacterial keratitis, corneal ulceration, phlyctenulosis, anterior uveitis, endophthalmitis, bacterial abscess, acute spetic retinitis, chronic bacterial retinitis, papillitis, optic neuritis, and orbital cellulitis.

The ophthalmic infection can be caused by numerous genera including but not limited to Staphylococcus, Streptococcus, Treponema, Pneumococcus, Gonococcus, Haemophilus, Klebsiella, Neisseria, Chlamydia, Mycobacterium, Flavobacterium, Serratia, Propionibacterium, Actinomyces, Pseudomonas, Corynebacterium, Meningococcus, and Enterococcus. The key species of the Staphylococcus and Streptococcus genera are Staphylococcus aureus, Staphylococcus epidermidis, Streptococcus viridans, and Streptococcus pneumoniae.

Viral conditions and infections of the eyes include but should not be limited to herpes zoster ophthalmicus, herpes simplex blepharitis, verruca, molluscum contagiosum, infective mononucleosis, viral conjunctivitis, episeleritis, scleritis, herpes simplex keratitis, iridocyclitis, ocular syphilis, cytomealovirus retinitis, and viral papillitis and optic neuritis. Also included in viral infections of the eyes are infections caused by poxvirus, herpetovirus, adenovirus, paramyxovirus and human immunodeficiency virus. The key species in herpetovirus are herpes simplex virus, herpes zoster virus, Epstein-Barr virus, and cytomegalovirus.

Fungal conditions and infections of the eyes include but are not limited to ringworm, fungal conjuctivitis, keratomycosis, uveitis, abscess, candida retinitis, fungal papillitis and optic neuritis, invasive aspergillosis, mucormycosis, microsporum, trichophyton, aspergillus, leptothrix, sporotrichum, fusarium, cephalosporium, cryptococcus, phycomycetes, and candida. The key species of aspergillus is aspergillus fumigatus. The key species of candida is candida albicans.

Vertebrates are the preferred animals contemplated for this invention. This includes mammals and non-mammals. Preferred mammals include but are not limited to Primates, Carnivora, Proboscidea, Perissodactyla, Artiodactyla, Rodentia and Lagomorpha. It is more preferred that the mammal be Canisfamiliaris (dog), Felis catus (cat), Elephas maximus (elephant), Equus caballus (horse), Sus domesticus (pig), Camelus dromedarius (camel), Cervus axis (deer), Giraffa camelopardalis (giraffe), Bos taurus (cattle), Capra hircus (goat), Ovis aries (sheep), Mus musculus (mouse), Lepus brachyurus (rabbit), Mesocricetus auratus (hamster), Cavia porcellus (guinea pig), Meriones unguiculatus (gerbil) and Homo sapiens (human). It is even more preferred that the mammal in the invention be Homo sapiens (human).

Another preferred embodiment of the invention is a method for treating an ophthalmic condition comprising administering to a vertebrate a peptide in a form consistent with the invention. The peptide of this invention is administered to the vertebrate through conjunctival administration, nasal administration, buccal administration, oral administration, rectal administration, vaginal administration, topical administration, and parenteral administration. The conjunctival administration refers to the delivery of the peptide across the corneal and conjunctival surface into the eye or the rest of the body of a vertebrate. The most common form of administration is an eye drop type delivery system. The nasal administration refers to the delivery of the peptide across the nasal mucous epithelium and into the peripheral circulation. Aerosolized sprays or topical applications are contemplated. The buccal administration refers to the delivery across the buccal or lingual epithelia into the peripheral circulation. Pastes, gels, dissolvable media and aerosolized sprays are contemplated. The oral administration refers to the delivery of the peptide through the buccal epithelia but predominantly swallowed and absorbed in the stomach and alimentary tract. The rectal administration refers to the delivery of the peptide via the lower alimentary tract via suppositories or enemas that bathe the mucosal membranes and cross into the peripheral circulation. The vaginal administration refers to the delivery of the peptide through the vaginal mucous membrane into the peripheral circulation. The epidermal administration refers to the delivery of the peptide across the dermis and absorption into the peripheral circulation. The parenteral administration refers to the injection of the peptide contained in a solution into the vertebrate. The injection in the parenteral administration can be intravenous, intramuscular, subcutaneous, subconjunctival, intraocular, retrobulbar, epidural, intramedullary, and intrathecal.

It is preferred that the peptide in this invention be administered into the vertebrate through a conjunctival administration where the peptide can be included in a form of an ophthalmic solution, an ophthalmic suspension, an ophthalmic gel, an ophthalmic ointment or an ophthalmic strip/insert. The ophthalmic solution is an aqueous or organic solution formulated to use as eye drops. The ophthalmic suspension is the addition of a small particle, e.g., microfine nano-particles, which contain the peptide, into an aqueous or organic solution. The ophthalmic gel is a special polymer that disperses in the tear film and forms an essentially transparent film across the ocular surface. The ophthalmic ointment is a mixture of a petrolatum base with wool fat. The ophthalmic strip/insert refers to a filter paper or insoluble contact lens-like object, which can be impregnated with the peptide. The impregnated ophthalmic strip/insert can then be placed onto the ocular surface or inserted into the lower cul-de-sac.

Another preferred embodiment of the invention is a method for treating an ophthalmic infection in a vertebrate using a therapeutically effective amount of a peptide. The therapeutically effective amount is at least 10⁻¹² Molar. It is preferred the therapeutically effective amount is at least about 10 ⁻⁸ Molar. The exact therapeutically effective amount depends on the particular administration being used, the age, weight, and general physical conditions of the particular vertebrate being treated, the severity of the ophthalmic infection, and other antimicrobial agents being used in combination with the peptide, as is well known to those skilled in the art.

Another preferred embodiment of the invention is a method for treating an ophthalmic infection in a vertebrate administering a therapeutically effective amount of a peptide. Since the peptide has antipyretic, anti-inflammatory, antibacterial, antifungal and antiviral properties, the peptide can be used immediately after the ophthalmic condition is observed before the insult causing the condition is determined. This is especially useful in treating infections where the specific infective entity is unknown. Further, the peptide can continuously be used after the insult causing the condition is determined.

Another preferred embodiment of the invention is a method for treating an ophthalmic infection in a vertebrate administering a therapeutically effective amount of a peptide before or for prevention of an eye condition. Further, the peptide can be used individually, or in combination with another peptide, or in combination with another antimicrobial agent, which is not a peptide, or in combination with a non-antimicrobial agent.

The following represents an example of a preferred formulation wherein the active ingredient, KPV, KPV-dimer or equivalent of KPV, is used in a eye drop formulation:

| | |
|---|---|
| Hydroxymethylcellulose | 0.2% |
| Sodium Chloride | 0.2% |
| Polysorbate 80 | 0.5% |
| Disodium edetate | 0.105% |
| Sodium phosphate, dibasic | 1.3% |
| Sorbic acid | 0.262% |
| Lecithin | .05% |
| Active Ingredient | 0.00285% |
| Sterile water | q.s. |

### EXAMPLES

### Example I

### Formation of the α-MSH Peptides and Derivatives Including KPV Dimer

The peptides used in the examples included: α-MSH (1-13) (SEQ. ID NO 4), α-MSH (SEQ. ID NO. 2) which is MEHFRWG, α-MSH (6-13) (SEQ. ID NO. 3), and α-MSH (11-13) (SEQ. ID NO. 1), all of which were N-acetylated and C-amindated, and ACTH (1-39) (SEQ. ID NO. 21) and ACTH (18-39) (SEQ. ID NO. 22) which is also called CLIP. The peptides were prepared by solid-phase peptide synthesis and purified by reversed-phase high performance liquid chromatography. Another peptide used in this research included a dimer of the amino acid sequence KPV (SEQ. ID NO. 1), specifically VPKCCKPV (SEQ. ID NO. 5), which also was N-acetylated and C-amidated (the “KPV dimer”). The VPKCCKPV (SEQ. ID NO. 5) can be chemically represented as Val-Pro-Lys-AcCys-s-s-CysAc-Lys-Pro-Val or VPKC-s-s-CKPV. The VPKCCKPV (SEQ. ID NO. 5) is formed by adding cysteines at the N-terminal of KPV (SEQ. ID NO. 1) peptide and allowing the cysteines of two CKPV peptide to form a disulfide bond. As shown in FIG. 9, the molecular conformation of the VPKC-s-s-CKPV (SEQ ID NO. 5) was studied through molecular modeling techniques. The molecular modeling studies were performed using the SYBYL software version 6.2 running on Silicon Graphic Indingo 2 workstation. The conformational study showed that the VPKCCKPV (SEQ. ID NO. 5) peptide adopts a like-β-turn-structure well organized and stabilized by intra-molecular hydrogen bounds. The tertiary structure of the dimer is folded and amino acids are well protected. It also resembles a cyclic peptide with a beta-turn.

### Example II

### The Peptides Inhibit HIV-p24 Expression in HIV Infected Cells

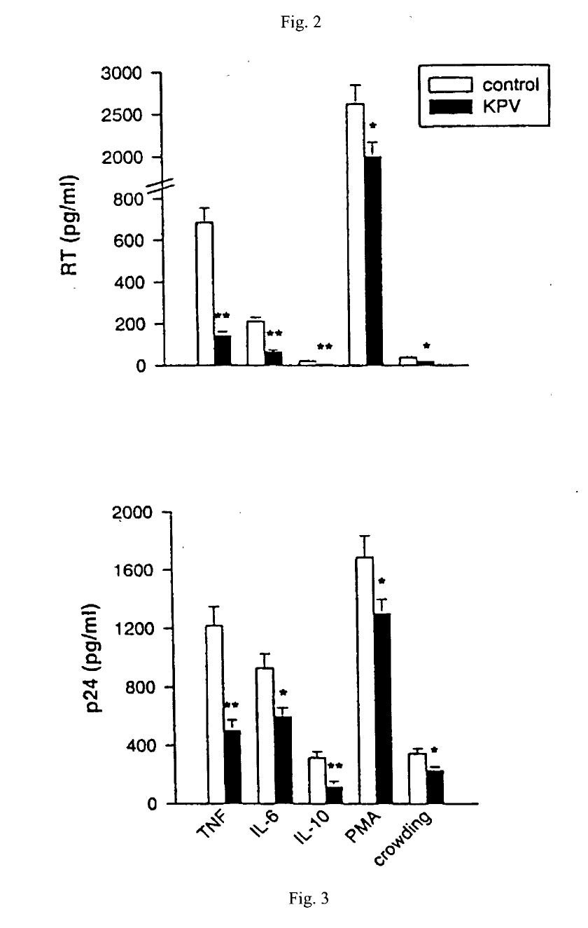

An HIV-1 infected promonocytic U1 cell line was maintained in complete culture medium (RPMI 1640 supplemented with 10 mM Hepes), 2 mM L-glutamine (Sigma-Aldrich), 10% heat-inactivated FCS (HyClone Laboratories, Logan, Utah, USA), penicillin at 100 units/mL and streptomycin at 100 μg/mL (Gibco Laboratories, Grand Island, N.Y.) in log phase of growth. Before use, cells were washed three times with HBSS (Gibco) to remove extracellular virus. Cells were plated onto 24-well flat-bottomed plates at a concentration of 2×10⁶/mL (final volume 1 mL) with medium plus TNF-α(10 ng/mL (R&D Systems, Oxford, England, UK) in the presence or absence of α-MSH peptides in concentrations from 10⁻¹³ to 10⁻⁴ M. Supernatants were removed by centrifugation after 48 hr incubation at 37° C. in 5% CO₂, and tested for HIV-p24 release. p24 antigen releases (Cellular Products Inc. Buffalo, N.Y., USA) were determined using commercial ELISA kits. In all experiments each condition was tested in triplicate. HIV-p24 is a capside HIV structure protein. The level of HIV-p24 reflects HIV infection and HIV viral amount. As shown in FIG. 2, α-MSH and the tripeptide KPV (SEQ. ID NO. 1) significantly inhibited p24 release from TNF-α-stimulated U1 cells. Inhibitory effects of α-MSH peptides occurred over a broad range of peptide concentrations including picomolar concentrations that occur in human plasma. Greater concentrations caused more pronounced HIV inhibition, with the most effective concentration for both peptides being 10⁻⁵ M. In this concentration, α-MSH (SEQ. ID NO. 4) and KPV (SEQ. ID NO. 1) caused 52.7% and 56.0% inhibition of p24 release, respectively.

### Example III

### The Peptides Inhibit HIV-p24 and Reverse Transcriptase Expression in HIV Infected Cells Stimulated by TNF-α, IL-6. IL-10, and PMA.

HIV-1 infected promonocytic U1 cells were plated onto 24-well flat-bottomed plates at a concentration of 2×10/mL (final volume 1 mL) with medium alone or TNF-α(10 ng/mL), IL-6 (20 ng/mL), IL-10 (20 ng/mL (R&D Systems) or PMA (1 ng/mL) (Sigma-Aldrich Chemicals, St. Louis, Mo., USA) in the presence or absence of KPV (SEQ. ID NO. 1) in concentrations of 10⁻⁵ M. Supernatants were removed by centrifugation after 48 hr incubation at 37° C. in 5% CO₂, and tested for HIV-p24 release and reverse transcriptase release. In crowding experiments, U1 cells were seeded at the density of 2×10⁵ mL and maintained in culture at 37° C. in 5% CO₂ without change of medium for 7 days. KPV (SEQ. ID NO. 1) in concentrations of 10⁻⁵M were added on day 1. p24 antigen releases (Cellular Products Inc. Buffalo, N.Y., USA) and reverse transcriptase (ELISA Retrosys RT assay, Innovagen, Lund, Sweden) were determined using commercial ELISA kits. In all experiments, each condition was tested in triplicate. As shown in FIG. 3, KPV (SEQ. ID NO. 1) significantly inhibited p24 and RT release from U1 cells induced by IL-6, IL-10, PMA, and in crowding conditions.

### Example IV

### The Peptides Inhibit HIV Transcription

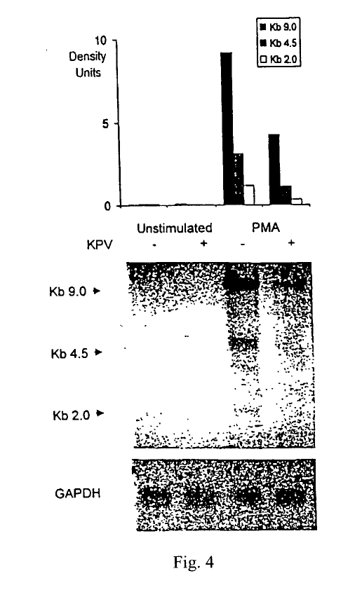

To determine the influence of KPV (SEQ. ID NO. 1) on HIV transcription, 2×10⁶ U1 cells (at a density of 2×10⁵/mL in complete medium) were stimulated for 24 h with PMA (1 ng/mL) in the presence or absence of KPV (SEQ. ID NO. 1)10⁻⁵M. Total RNA was extracted by the guanidine thiocyanate phenol method using an RNA isolation kit (Tripure, Boehringer Mannheim, Indianapolis, Ind.), following the manufacturer's instructions. Ten μg of total RNA were separated by 0.8% agarose/formaldehyde gel electrophoresis and transferred onto nylon membrane. The filters were baked and hybridized for 18 hr with α³² P-labeled HIV-full length probe (kind gift of L. Turchetto and E. Vicenzi, S. Raffaele Hospital, Milan, Italy). The radiolabeling reaction was performed using a DNA labeling kit (Ready-to-go, Pharmacia Biotech, San Francisco, Calif.). Filters were washed and exposed to X-ray film for 5 days. The labeled probe was removed by washing at 80° C. in 0.1×SSC containing 0.1% sodium dodecyl sulphate and then rehybridized with α³² P-labeled glyceraldehyde-3-phosphate dehydrogenase (GAPDH) cDNA probe. Densitometric analysis was performed using ImageMaster™ VDS 3.0 software (Pharmacia Biotech) and results were expressed as density units. As shown in FIG. 4, the inhibitory activity of KPV (SEQ. ID NO. 1) on HIV transcription was confirmed by Northern blot analysis of HIV-RNA in PMA-stimulated U1 cells. Addition of KPV (SEQ. ID NO. 1) reduced by approximately 50% both spliced and unspliced HIV-1 RNA in PMA-stimulated U1 cells.

### Example V

### The Peptides Severely Decrease the Viability of Staphylococcus aureus

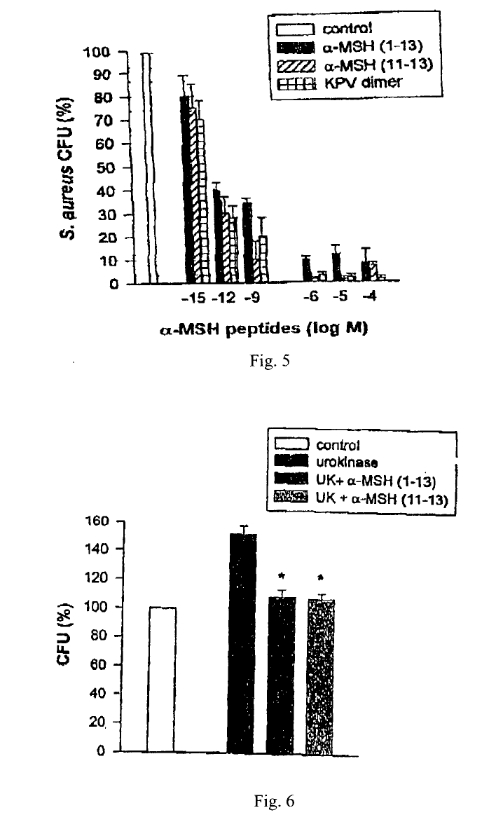

Staphylococcus aureus (ATCC29213) was obtained from the collection of the Department of Microbiology, Ospedale Maggiore di Milano. S. aureus (1×10⁶/ml in HBSS) was incubated in the presence or absence of α-MSH [1-13] (SEQ. ID NO 4), α-MSH [11-13] (SEQ. ID NO. 1) or the “KPV dimer” (SEQ. ID NO. 5) at concentrations in the range of 10⁻¹⁵ to 10⁴M for 2 hours at 37° C. S. aureus were then washed in cold distilled water and diluted with HBSS to a concentration of 100 organisms/ml. One ml aliquots were dispensed on blood agar plates and incubated for 24 hours at 37° C. Organism viability was estimated from the number of colony forming units. As shown in FIG. 5, α-MSH [1-13] (SEQ. ID NO. 4) and α-MSH [11-13] (KPV) (SEQ. ID NO. 1) inhibited S. aureus colony formation. The KPV dimer (SEQ. ID NO. 5) also inhibited S. aureus colony formation. The inhibitory effect occurred over a wide range of concentrations and was statistically significant (p<0.01) with peptide concentrations of 10¹² to 10⁴M.

### Example VI

### The Peptides Severely Decrease the Viability of Urokinase-Induced Growth-Enhanced Staphylococcus aureus

In this experiment, the influence of α-MSH on urokinase-induced growth-enhancement is determined. Hart, D. A.; Loule, T.; Krulikl, W.; Reno, C., Staphylococcus Aureus Strains Differ in Their in Vitro Responsiveness to Human Urokinase: Evidence that Methicillin-Resistant Strains are Predominantly Nonresponsive to the Growth-Enhancing Effects of Urokinase, Can. J. Microbiol. 42, 1024-31 (1966). S. aureus (10⁵/100 ml) were incubated for four hours at 37° C. with recombinant human urokinase 500 U (Lepetit, Milan, Italy) in a shaking water bath, in the presence or absence of α-MSH [1-13] (SEQ. ID NO. 4) or α-MSH [11-13] (SEQ. ID NO. 1) at 10⁻⁶ M. Appropriate dilutions of S. aureus were dispensed on agar plates and colonies counted after 24 hours incubation at 37° C. As shown in FIG. 6, the treatment with urokinase increased S. aureus colony formation and addition of α-MSH [1-13] (SEQ. ID NO. 4) or α-MSH [11-13] which is KPV (SEQ. ID NO. 1) at concentrations of 10⁻⁶M significantly inhibited the enhancing effect of urokinase.

### Example VII

### The Peptides Severely Decreases the Viability of Candida albicans

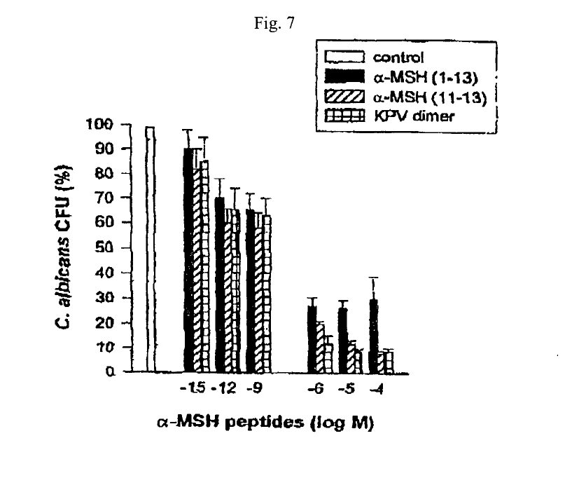

C. albicans (clinical isolate) were obtained from the collection of the Department of Microbiology, Ospedale Maggiore di Milano. C. albicans were maintained on Sabouraud's agar slants and periodically transferred to Sabouraud's agar plates and incubated for 48 hours at 28° C. To prepare stationary growth phase yeast, a colony was taken from the agar plate and transferred into 30 ml Sabouraud-dextrose broth and incubated for 72 hours at 32° C. Cells were centrifuged at 100×g for 10 minutes and the pellet was washed twice with distilled water. Cells were counted and suspended in Hank's balanced salt solution (“HBSS”) to the desired concentration. Viability, determined by the exclusion of 0.01% methylene blue, remained >98%. C. albicans was then (1×10⁶/ml in HBSS) was incubated in the presence or absence of α-MSH [1-13], α-MSH [11-13] which is KPV, (SEQ. ID NO. 1) or the “KPV dimer” (SEQ. ID NO. 5) at concentrations in the range of 10⁻¹⁵ to 10⁻⁴M for 2 hours at 37° C. Cells were then washed in cold distilled water and diluted with HBSS to a concentration of 100 organisms/ml. One ml aliquots were dispensed on blood agar plates and incubated for 48 hours at 37° C. Organism viability was estimated from the number of colonies formed. As shown in FIG. 7, C. albicans colony forming units were greatly reduced by α-MSH and KPV. (SEQ. ID NO. 1) A dimer of the amino acid sequence KPV, specifically, VPKCCKPV (SEQ. ID NO. 5) also inhibited C. albicans colony formation. Concentrations of all three peptides from 10⁻¹³ to 10⁻⁴M had significant inhibitory influences on CFU (p<0.01 vs. control).

### Example VIII

### Potency of Among the Peptides in Reducing C. albicans Viability in Comparison with Fluconazole and ACTH

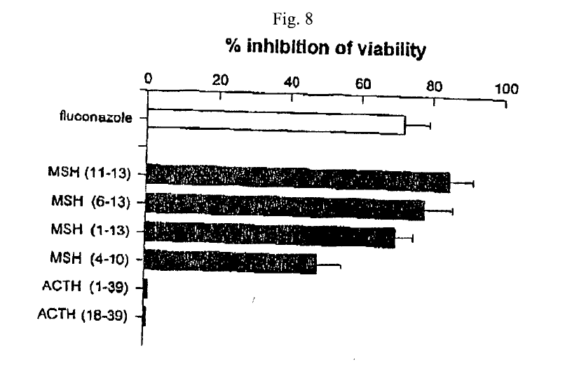

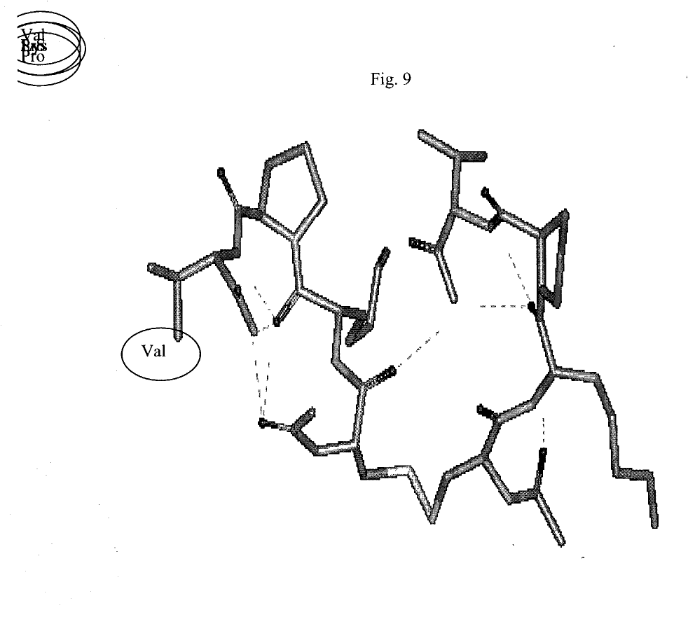

Fluconazole is a well established antifungal agent. The potency of the peptides in reducing C. albicans viability is studied in comparison with fluconazole and ACTH using similar procedures as in Example VII. The peptides and fluconazole were tested in concentrations of 10⁶ M. There were at least six replicates for each concentration of peptide. As shown in FIG. 8, α-MSH [11-13] (KPV), (SEQ. ID NO. 1) α-MSH [6-13] (SEQ. ID NO. 3), and α-MSH [1-13] (SEQ. ID NO. 4) were the most effective. Their inhibitory activity was similar to that of fluconazole. The “core” α-MSH sequence, α-MSH [4-10] (SEQ. ID NO. 2) caused approximately 50% inhibition of CFU. Although this inhibitory effect was substantial (p<0.01. vs. control), it was significantly less than that caused by α-MSH fragments bearing the KPV signal sequence, i.e., α-MSH [6-13] (SEQ. ID NO. 3) and α-MSH [11-13] (SEQ. ID NO. 1) (p<0.01), or the parent molecule α-MSH [1-13] (SEQ. ID NO. 4) (p<0.05). ACTH (1-39) (SEQ. ID NO. 21) and the ACTH fragment (18-39) (SEQ. ID NO. 22) did not reduce C. albicans viability (FIG. 4). Even higher concentrations of these ACTH peptides (up to 10⁻⁴ M) were likewise ineffective in reducing C. albicans CFU (results not shown in the figures).

### Example IX

### Treatment of Viral Ophthalmic Infection—HSV Blepharitis

A patient is diagnosed as having Herpes simplex virus blepharitis. A ophthalmic aqueous solution containing 10⁻⁵ M of KPV (SEQ. ID NO. 1) is prepared. The patient is treated with 3-5 drops of the ophthalmic aqueous solution onto the surface of eyes four times daily for 7 days. After 7 days of treatment, the HSV blepharitis symptom is substantially reduced.

### Example X

### Treatment of Bacterial Ophthalmic Infection—Bacterial Keratitis

A patient presents complaining of eye pain when blinking and blurred vision. Upon examination the cornea appears subtly less transparent than normal cornea and may have ulcers on its surface. The diagnosis is infectious keratitis of bacterial etiology, which is confirmed by laboratory findings. The patient is treated with 3-5 drops of the ophthalmic aqueous solution containing 10⁻⁵ M of KPV (SEQ. ID NO. 1) onto the surface of eyes four times daily for 7 days. After 7 days of treatment, the patient either shows marked improvement or fully recovers.

### Example XI

### Treatment of Fungal Ophthalmic Infection—Keratomycosis

Upon examination, a patient is diagnosed as having Keratomycosis. The patient is treated with 3-5 drops of the ophthalmic aqueous solution containing 10⁻⁵ M of KPV (SEQ. ID NO. 1) onto the surface of eyes four times daily for 7 days. After 7 days of treatment, the patient either shows marked improvement or fully recovers.

### Example XII

### Treatment of Ophthalmic Infection Before Microorganism Causing the Infection is Determined

A patient presents complaining of reddened and swollen eyelids and the presence of mucoid secretions on the eye which interfere with vision. While awaiting the results of the culture and sensitivity based on the mucoid sample, the patient is treated with 3-5 drops of the ophthalmic aqueous solution onto the surface of eyes four times daily for 3 days and symptoms disappears. The laboratory tests later show that the patient is inflicted with bacterial conjunctivitis.

### Example XIII

### Treatment of CVS or “Computer Eyes”

An office worker may spend nearly 5-7 hours in front of a computer screen or other visual monitor. After a certain length of time the worker may begin to blink less. Within a short period thereafter, the worker may notice a dryness of the eyes, burning and blurred vision. The worker may choose to seek medical help, fearing that there may not be time to take work off to have a medical professional access the workers eye condition. In this event, the worker purchases an over the counter preparation of eye drops containing the claimed invention. Upon usage of the eye drops the worker's eye condition may be substantial relieved. Further, the eye drops may be repeated without fear of injury to the eyes, as the active ingredients of the eye drops are natural.

### Example XIV

### Comparison of Symptoms Before and After Treatment with Invention

The following example is presented in table format. Symptoms were compared with use of the invention and with symptoms when the invention had not been used. The study contained 22 subjects with varying symptoms treated with the invention. The study shows that the invention may be used for successfully for a variety of symptoms associated with pathologic conditions of the eyes.

TABLE 1
Treatment Study

(n = 22)

| | | |
|---|---|---|
| Age, years | 34 | (9.9)¹ |
| Contacts | 3 | (14%)² |
| Eyeglasses | 8 | (36%) |
| Ever used artifical tears | 3 | (14%) |
| Hours per day spent before computer | 8.0 | (1.3) |
| screen | | |
| Used OTC medication for eye problems in | 2 | (9%) |
| last month | | |
| Used presciption to treat dry eye, allergies | 1 | (5%) |
| or other disorders in the lasy 6 months | | |
| Self reported near vision | | |
| Normal/good | 17 | (77%) |
| Very good | 4 | (18%) |
| Poor | 1 | (5%) |
| Comfort level before using drops | | |
| Very uncomfortable | 3 | (14%) |
| Uncomfortable | 5 | (23%) |
| Slightly uncomfortable | 7 | (32%) |
| Comfortable | 5 | (23%) |
| Very comfortable | 2 | (9%) |
| Comfort level after using drops | | |
| Slightly uncomfortable | 2 | (9%) |
| Comfortable | 15 | (68%) |
| Very comfortable | 5 | (23%) |
| Times per day used drops | 2.3 | (0.6) |
| Drops used per application | 2.6 | (0.9) |
| Would use this product if it becomes | 20 | (91%) |
| available over the counter | | |

¹Mea, (sd);
²Frequency (percent)

TABLE 2
Comparison of Treatment Invention of No Treatment

| | | Comparison to day one |
|---|---|---|
| | Total Score | before treatment |
| | Mean (sd) | Paired t-test p-value |
| Day one-before treatment | 7.5 (6.2) | |
| Day one-5 minutes after | 3.2 (5.0) | <0.0001 |
| 1^{st} application | | |
| Day one-5 minutes after | 2.4 (3.8) | 0.0003 |
| 2^{nd} application | | |
| Day one-end of the day | 1.6 (2.6) | <0.0001 |
| Day two-before treatment | 3.9 (4.8) | 0.0001 |
| Day two-5 minutes after | 2.3 (3.2) | <0.0001 |
| 1^{st} application | | |
| Day two-5 minutes after | 1.5 (2.3) | <0.0001 |
| 2^{nd} application | | |
| Day two-end of the day | 0.8 (1.3) | 0.0001 |

TABLE 3
Prevalance of individual symptoms

| | | Day 1 | Day 1 | | | Day 2 | Day 2 | |
|---|---|---|---|---|---|---|---|---|
| | Day 1 | 5 min. | 5 min. | Day 1 | Day 2 | 5 min. | 5 min. | Day 2 |
| | before | 1^{st} | 2^{nd} | end of | before | 1^{st} | 2^{nd} | end of |
| | treatment | application | application | the day | treatment | application | application | the day |
| Blurring | 73% | 27% | 18% | 23% | 27% | 18% | 14% | 9% |
| Burning | 23% | 32% | 27% | 23% | 14% | 23% | 18% | 14% |
| Crusting | 9% | 5% | 9% | 9% | 9% | 5% | 5% | 0 |
| Dryness | 82% | 41% | 23% | 32% | 59% | 27% | 18% | 18% |
| Foreign body | 14% | 9% | 9% | 5% | 14% | 5% | 0 | 0 |
| sensation | | | | | | | | |
| Onvoluntary | 18% | 14% | 9% | 5% | 9% | 14% | 5% | 0 |
| jumping of eye | | | | | | | | |
| Pain | 36% | 18% | 18% | 9% | 18% | 18% | 14% | 5% |
| Redness | 50% | 36% | 23% | 18% | 27% | 36% | 32% | 18% |
| Scratchy | 27% | 18% | 14% | 14% | 14% | 18% | 9% | 9% |
| Sensitivity to | 59% | 14% | 14% | 5% | 41% | 27% | 14% | 9% |
| bright light | | | | | | | | |

From the foregoing, it will be appreciated that specific embodiments of the invention have been described herein for purposes of illustration, but that various modifications may be made without deviating from the spirit and scope of the invention. Accordingly, the invention is not limited except as by the appended claims. The preceding Examples are intended only as examples and are not intended to limit the invention. It is understood that modifying the examples above does not depart from the spirit of the invention. It is further understood that the each example may be applied on its own or in combination with other examples.

What is claimed is:

1. A pharmaceutical composition consisting of hydroxyethylcellulose (0.2%), sodium chloride (0.2%), polysorbate 80 (0.5%), disodium edetate (0.105%), sodium phosphate, dibasic (1.3%), sorbic acid (0.262%), lecithin (0.05%), α-MSH peptides (0.00285%) and sterile water.

2. The pharmaceutical composition of claim 1 wherein the α-MSH peptides are selected from the group consisting of KPV (SEQ. ID NO. 1), MEHFRWG (SEQ. ID NO. 2), HFRWGKPV (SEQ. ID NO. 3), SYSMEHFRWGKPV (SEQ. ID NO. 4), a KPV dimer (SEQ. ID NO. 5), and biologically functional equivalents thereof.

3. The pharmaceutical composition of claim 2 wherein the KPV dimer may be modified.

4. The pharmaceutical composition of claim 3 wherein the modified KPV dimer is selected from the group consisting of VPK-Cys-s-s-Cys-KPV (SEQ. ID NO. 5), VPK-DCys-s-s-Cys-KPV (SEQ. ID NO. 6), VPK-Pen-s-s-Cys-KPV (SEQ. ID NO. 7), VPK-Pen-s-s-DCys-KPV (SEQ. ID NO. 8), VPK-DPen-s-s-Cys-KPV (SEQ. ID NO. 9), VPK-DPen-s-s-DCys-KPV (SEQ. ID NO. 10), VPK-DPen-s-s-DPen-KPV (SEQ. ID NO. 11), VPK-Pen-s-s-Pen-KPV (SEQ. ID NO. 12), VPK-hCys-s-s-Cys-KPV (SEQ. ID NO. 13), VPK-hCys-s-s-DCys-KPV (SEQ. ID NO. 14), VPK-hCys-s-s-hCys-KPV (SEQ. ID NO. 15), VPK-DhCys-s-s-DhCys-KPV (SEQ. ID NO. 16), VPK-DhCys-s-s-hCys-KPV (SEQ. ID NO. 17), VPK-hCys-s-s-Pen-KPV (SEQ. ID NO. 18), VPK-hCys-s-s-DPen-KPV (SEQ. ID NO. 19), and VPK-DhCys-s-s-DPen-KPV (SEQ. ID NO. 20).

5. A method of treating conditions of the eye comprising using an effective amount of the pharmaceutical composition of claim 4.

6. The method of treating eye conditions of claim 5 wherein the eye condition may be selected from the group consisting of blepharitis, hordeolum, preseptal cellulitis, dacryocystitis, orbital cellulitis, erysipelas, vernal keratoconjunctivitis, bacterial conjunctivitis, conjunctival laceration, superior limbic keratoconjunctivitis, conjunctivitis with pseudomembrane, epidemic keratoconjunctivitis, bacterial keratitis, corneal ulceration, phlyctenulosis, anterior uveitis, endophthalmitis, bacterial abscess, acute spetic retinitis, chronic bacterial retinitis, papillitis, optic neuritis, and orbital cellulitis, xerosis, computer vision syndrome, eye inflammation, computer eyes, and eyestrain and fatigue.

7. The method of treating conditions of the eye of claim 5 wherein the effective amount is 1-3 drops in each eye every 3 hours.

8. The effective amount of claim 7 wherein the method of treatment may be before, during, and/or after an attack of an eye condition.

9. A method of treating an ophthalmic conditions comprising administering a therapeutically effective amount of an α-MSH peptide in a vertebrate whereas the vertebrate is inflicted with the ophthalmic infection.

10. The method of treating an ophthalmic infection of claim 9 wherein the peptide is selected from the group consisting of KPV (SEQ. ID NO. 1), MEHFRWG (SEQ. ID NO. 2), HFRWGKPV (SEQ. ID NO. 3), SYSMEHFRWGKPV (SEQ. ID NO. 4), a KPV dimer (SEQ. ID NO. 5), and a biologically functional equivalent thereof.

11. The method of treating an ophthalmic infection according to claim 9 wherein the KPV dimer may be modified.

12. The method of treating an ophthalmic infection according to claim 11 wherein the modified KPV dimer is selected from the group consisting of VPK-Cys-s-s-Cys-KPV (SEQ. ID NO. 5), VPK-DCys-s-s-Cys-KPV (SEQ. ID NO. 6), VPK-Pen-s-s-Cys-KPV (SEQ. ID NO. 7), VPK-Pen-s-s-DCys-KPV (SEQ. ID NO. 8), VPK-DPen-s-s-Cys-KPV (SEQ. ID NO. 9), VPK-DPen-s-s-DCys-KPV (SEQ. ID NO. 10), VPK-DPen-s-s-DPen-KPV (SEQ. ID NO. 11), VPK-Pen-s-s-Pen-KPV (SEQ. ID NO. 12), VPK-hCys-s-s-Cys-KPV (SEQ. ID NO. 13), VPK-hCys-s-s-DCys-KPV (SEQ. ID NO. 14), VPK-hCys-s-s-hCys-KPV (SEQ. ID NO. 15), VPK-DhCys-s-s-DhCys-KPV (SEQ. ID NO. 16), VPK-DhCys-s-s-hCys-KPV (SEQ. ID NO. 17), VPK-hCys-s-s-Pen-KPV (SEQ. ID NO. 18), VPK-hCys-s-s-DPen-KPV (SEQ. ID NO. 19), and VPK-DhCys-s-s-DPen-KPV (SEQ. ID NO. 20).

13. The method of claim 9 wherein the ophthalmic condition is selected from the group consisting of a bacterial ophthalmic infection, a fungal ophthalmic infection, and a viral ophthalmic infection.

14. The method of claim 13 wherein the bacterial ophthalmic infection is caused by a Staphylococcus, Streptococcus, Treponema, Pneumococcus, Gonococcus, Haemophilus, Klebsiella, Neisseria, Chlamydia, Mycobacterium, Flavobacterium, Serratia, Propionibacterium, Actinomyces, Pseudomonas, Corynebacterium, Meningococcus, and Euterococcus.

15. The method of claim 14 wherein the bacterial ophthalmic infection is caused by either Staphylococcus or Streptococcus.

16. The method of claim 15 wherein the bacterial ophthalmic infection is caused by Staphylococcus aureus, Staphylococcus epidermidis, Streptococcus viridans, or Streptococcus pneumoniae.

17. The method of claim 13 wherein the fungal ophthalmic infection is caused by a Microsporum, Trichophyton, Aspergillus, Leptothrix, Sporotrichum, Fusarium, Cephalosporium, Cryptococcus, Phycomycetes, or Candida.

18. The method of claim 17 wherein the fungal ophthalmic infection is caused by Aspergillus or Candida.

19. The method of claim 18 wherein the fungal ophthalmic infection is caused by Aspergillus fumigatus or Candida albicans.

20. The method of claim 13 wherein the viral ophthalmic infection is caused by a Poxvirus, a Herpesvirus, an Adenovirus, a Paramyxovirus or HIV.

21. The method of claim 21 wherein the viral ophthalmic infection is caused by a HIV, a Herpes simplex virus, a Herpes zoster virus, an Epstein-Barr virus, or a Cytomegalovirus.

22. The method of claim 9 wherein the vertebrate is a bird or a mammal.

23. The method of claim 22 wherein the bird is a Columba livia, a Gallus domesticus, or a Meleagris gallopavo.

24. The method of claim 22 wherein the mammal is a Primate, a Carnivora, Proboscidea, a Perissodactyla, an Artiodactyla, a Rodentia, or a Lagomorpha.

25. The method of claim 24 wherein the mammal is a Canis familiaris, a Felis catus, an Elephas maximus, an Equus caballus, a Sus domesticus, a Camelus dromedarius, a Cervus axis, a Giraffa camelopardalis, a Bos taurus, a Capra hircus, an Ovis aries, a Mus musculus, a Lepus brachyurus, a Mesocricetus auratus, a Cavia porcellus, a Meriones unguiculatus, or a Homo sapiens.

26. The method of claim 25 wherein the mammal is a Homo sapiens.

27. The method of claim 9 wherein the peptide is administered through a conjunctival administration, a nasal administration, a buccal administration, an oral administration, a rectal administration, a vaginal administration, an epidermal administration, or a parenteral administration.

28. The method of claim 27 wherein the peptide is administered through a conjunctival administration.

29. The method of claim 28 wherein the peptide is administered through the conjunctival administration in a form of a ophthalmic solution, an ophthalmic suspension, an ophthalmic gel, an ophthalmic ointment, or an ophthalmic strip/insert.

30. The method of claim 29 wherein the ophthalmic solution is comprised of hydroxyethylcellulose (0.2%), sodium chloride (0.2%), polysorbate 80 (0.5%), disodium edetate (0.105%), sodium phosphate, dibasic (1.3%), sorbic acid (0.262%), lecithin (0.05%), any of the peptides of claim 2-4 (0.00285%) and sterile water.

31. The method of claim 9 wherein the peptide is administered before, during or after a cause of the ophthalmic condition is determined.

32. The method of claim 9 wherein the therapeutically effective amount is at least 10⁻¹³ Molar.

33. The method of claim 9 wherein the therapeutically effective amount is at least 10⁻⁸ Molar.

34. A method of treating an ophthalmic infection comprising administering a therapeutically effective amount of a peptide in Homo sapiens with an ophthalmic infection.

35. The method of claim 34 wherein the peptide is selected from the group consisting of KPV (SEQ. ID NO. 1), MEHFRWG (SEQ. ID NO. 2) HFRWGKPV (SEQ. ID NO. 3), SYSMEHFRWGKPV (SEQ. ID NO. 4), a KPV dimer (SEQ. ID NO. 5), and a biologically functional equivalent thereof.

36. The method of claim 35 wherein the KPV dimer may be modified.

37. The method of treating an ophthalmic infection according to claim 36 wherein the modified KPV dimer may be selected from the group consisting of VPK-Cys-s-s-Cys-KPV (SEQ. ID NO. 5), VPK-DCys-s-s-Cys-KPV (SEQ. ID NO. 6), VPK-Pen-s-s-Cys-KPV (SEQ. ID NO. 7), VPK-Pen-s-s-DCys-KPV (SEQ. ID NO. 8), VPK-DPen-s-s-Cys-KPV (SEQ. ID NO. 9), VPK-DPen-s-s-DCys-KPV (SEQ. ID NO. 10), VPK-DPen-s-s-DPen-KPV (SEQ. ID NO. 11), VPK-Pen-s-s-Pen-KPV (SEQ. ID NO. 12), VPK-hCys-s-s-Cys-KPV (SEQ. ID NO. 13), VPK-hCys-s-s-DCys-KPV (SEQ. ID NO. 14), VPK-hCys-s-s-hCys-KPV (SEQ. ID NO. 15), VPK-DhCys-s-s-DhCys-KPV (SEQ. ID NO. 16), VPK-DhCys-s-s-hCys-KPV (SEQ. ID NO. 17), VPK-hCys-s-s-Pen-KPV (SEQ. ID NO. 18), VPK-hCys-s-s-DPen-KPV (SEQ. ID NO. 19), and VPK-DhCys-s-s-DPen-KPV (SEQ. ID NO. 20).

38. The method of claim 34 wherein the ophthalmic infection is selected from the group consisting of a bacterial ophthalmic infection, a fungal ophthalmic infection, and a viral ophthalmic infection.

39. The method of claim 38 wherein the bacterial ophthalmic infection is caused by Staphylococcus, Streptococcus, Treponema, Pneumococcus, Gonococcus, Haemophilus, Klebsiella, Neisseria, Chlamydia, Mycobacterium, Flavobacterium, Serratia, Propionibacterium, Actinomyces, Pseudomonas, Corynebacterium, Meningococcus, or Euterococcus.

40. The method of treating an ophthalmic infection according to claim 39 wherein the bacterial ophthalmic infection is caused by Staphylococcus or Streptococcus.

41. The method of treating an ophthalmic infection according to claim 40 wherein the bacterial ophthalmic infection is caused by Staphylococcus aureus, Staphylococcus epidermidis, Streptococcus viridans, or Streptococcus pneumoniae.

42. The method of treating an ophthalmic infection according to claim 34 wherein the fungal ophthalmic infection is caused by Microsporum, Trichophyton, Aspergillus, Leptothrix, Sporotrichum, Fusarium, Cephalosporium, Cryptococcus, Phycomycetes, or Candida.

43. The method of claim 42 wherein the fungal ophthalmic infection is caused by Aspergillus or Candida.

44. The method of claim 43 wherein the fungal ophthalmic infection is caused by Aspergillus fumigatus or Candida albicans.

45. The method of treating an ophthalmic infection according to claim 34 wherein the viral ophthalmic infection is caused by a Poxvirus, a Herpesvirus, an Adenovirus, a Paramyxovirus or a HIV.

46. The method of treating an ophthalmic infection according to claim 45 wherein the viral ophthalmic infection is caused by a HIV, a Herpes simplex virus, a Herpes zoster virus, an Epstein-Barr virus, or a Cytomegalovirus.

47. The method of treating an ophthalmic infection according to claim 46 wherein the peptide is administered through a conjunctival administration, a nasal administration, a buccal administration, an oral administration, a rectal administration, a vaginal administration, an epidermal administration, or a parenteral administration.

48. The method of treating an ophthalmic infection according to claim 47 wherein the peptide is administered through a conjunctival administration.

49. The method of treating an ophthalmic infection according to claim 48 wherein the peptide is administered through the conjunctival administration in a form of a ophthalmic solution, an ophthalmic suspension, an ophthalmic gel, an ophthalmic ointment or an ophthalmic strip/insert.

50. The method of treating an ophthalmic infection according to claim 49 wherein the ophthalmic solution is comprised of hydroxyethylcellulose (0.2%), sodium chloride (0.2%), polysorbate 80 (0.5%), disodium edetate (0.105%), sodium phosphate, dibasic (1.3%), sorbic acid (0.262%), lecithin (0.05%), any of the peptides of claim 2-4 (0.00285%) and sterile water.

51. The method of treating any of the ophthalmic infections of claim 44 wherein the peptide is administered before, during or after a cause of the ophthalmic infection is determined.

52. A method of treating an ophthalmic infection according to claim 26 wherein the effective ophthalmologically amount is at least 10⁻¹³ Molar.

53. A method of treating an ophthalmic infection according to claim 26 wherein the effective ophthalmologically amount at least 10⁻⁸ Molar.
